(19) United States
(12) Patent Application Publication
(10) Pub. No.: US 2006/0053652 A1
(43) Pub. Date: Mar. 16, 2006

(54) Freeze-drying microscope stage apparatus and process of using the same

(76) Inventors: J Richard Gyory, Sudbury, MA (US); Javier Gonzalez-Zugasti, North Billerica, MA (US); David Putnam, Ithaca, NY (US)

Correspondence Address: TRANSFORM PHARMACEUTICALS, INC., 29 HARTWELL AVENUE, LEXINGTON, MA, 02421, US

(21) Appl. No.: 10/534,596

(22) PCT Filed: Nov. 20, 2003
(86) PCT No.: PCT/US03/37518

§ 371 Date: May 11, 2005

Related U.S. Application Data
(60) Provisional application No. 60/428,170, filed on Nov. 21, 2002.

(51) Int. Cl. F26B 5/06; C12Q 1/00

(52) U.S. Cl. 34/284; 435/4

(57) ABSTRACT

The invention concerns methods, systems, and devices for screening arrays comprising hundreds, thousands, or hundreds of thousands of samples. These methods are useful to optimize, select, and discover compositions, or conditions for cost-effective freeze-drying of preparations and freezing of biologicals while maintaining structural integrity and/or viability. Such freeze-dried compositions are easily reformulated for treating or preventing diseases, the cause of the diseases, or the symptoms of the diseases. Moreover, optimized freezing of biological samples enables viable preservation of a wide variety of biologicals.

### FIELD OF THE INVENTION

This invention is directed to the generation and analysis of data concerning freezing and freeze-drying.

### BACKGROUND OF THE INVENTION

Preserving biological matter and chemicals by reducing the temperature is an economically important process. In addition, the possibility of freezing live biological specimens for maintaining viability over long periods of time is another invaluable strategy for preserving tissues and cells. Freeze-drying substances and specimens in a frozen or solid state to remove volatile solvents and liquids has expanded these processes. Freeze-drying is one of the processes typically used for removing water from a preparation, usually to enhance preservation and/or reconstitution. The resulting residue is preferably stable even at room temperatures and is preferably easily reconstituted.

Not surprisingly, such processing neatly dovetails into discovery of pharmaceutical formulations that optimize stability, storage, bioavailability, and duration of action of one or more pharmaceuticals of interest and minimizes undesirable properties. Pharmaceuticals are rarely distributed as pure compounds for reasons of, among others, stability, solubility, and bioavailability. Furthermore, it is of interest to determine methods of economically preparing biological specimens in particular and specimens of interest in general for preservation.

However, discovering conditions for freeze-drying substances or long-term preservation of viable or even structurally faithful biological specimens is a tedious and time consuming task that limits the use of freezing and freeze-drying. Thus, it is of interest to determine methods of economically preparing pharmaceutical formulations that are suitable for storage with long-term stability and are readily reconstituted.

Freeze-drying requires significant energy and time overheads that are tedious due to the strong dependence of time on various parameters. Some of the parameters are the temperature, heat flow, pressure, the relative amounts of vitrified water and crystallized water, and the particular choice of excipient and processing procedure, such as the method of freezing the material. Typically, in the case of compositions, freeze-drying is performed in vials directly followed by sealing and packaging. Reconstitution of the composition in the vial then requires little more than adding the desired solvent. On the other hand, errors in maintaining proper freeze-drying conditions may result in a product that rather than being easy to dissolve/reconstitute or thaw, is instead collapsed and/or seriously damaged.

Essentially, freeze-drying requires removal of moisture or other solvent(s) via sublimation from a preparation maintained in a solid-phase by subjecting it to a suitable temperature and pressure. Sublimation is preferable to evaporation for removing one or more solvents from a liquid phase because during evaporation surface tension interferes with the preservation of the structure of specimens and the ability to ensure that the residue is easily reconstituted. Moreover, as solutes become more concentrated with the removal of water undesirable chemical changes occur more readily in the liquid phase. Thus, avoiding the liquid phase during removal of water also reduces undesirable chemical changes.

Freeze-drying typically comprises two or more phases. In the primary phase of freeze-drying, crystallized or otherwise sequestered solvent is removed from a preparation maintained in a solid state by application of suitable temperature and pressure conditions. Following this long primary phase, further removal of frozen solvent molecules in a vitrified state is carried out in a secondary-drying phase until the preparation exhibits desirable properties. The two phases are not always carried out in distinct steps since primary drying methods may be suitable for secondary drying as well.

Removal of solvents and volatile components enables superior preservation of structure due to a lack of the deleterious effects of surface tension on delicate structures. In addition, in the case of dissolved non-volatile solutes, subsequent reconstitution is faster and easier than with most other techniques due to the preservation of a large surface area in the solute left behind as the frozen solvent is removed. Importantly, a freeze-dried preparation is often significantly more stable at a higher temperature, such as room temperature. Moreover, in many circumstances there is a significantly lower possibility of contamination by, and growth, of microorganisms in freeze-dried preparations. These properties reduce storage and transportation costs and allow an otherwise labile active-component to be used in inhospitable environments lacking adequate refrigeration or similar facilities.

These properties, individually or in combination, are useful in making products ranging from pharmaceuticals to tissue specimens. For instance, pharmaceuticals are typically administered in a pharmaceutical formulation comprising one or more active ingredients and excipients. Physical and chemical properties, such as stability, solubility, dissolution, permeability, and partitioning of most pharmaceuticals are directly related to the medium in which they are administered. This is because the medium affects the physical and chemical environment of the active ingredient, e.g., a pharmaceutical. Moreover, the processing of the pharmaceutical formulation by various processes, such as freeze-drying, depends on the excipients as well. Excipients have an effect on the physical and chemical properties of pharmaceutical formulation mixtures upon administration to a patient, such as absorption, bioavailability, metabolic profile, toxicity, and potency. Such effects are caused by physical and chemical interactions between the excipients and the pharmaceutical and/or physical and chemical interactions between the excipients themselves.

Thus a goal of formulation development is to discover formulations that optimize desired characteristics of a pharmaceutical, such as stability, solubility, reconstitution, and bioavailability of the pharmaceutical. This is normally a tedious process, where each variable is separately assessed, at several points over a range of conditions or combinations. For example, if the formulation contains a pharmaceutical characterized by poor solubility, the solubility of the pharmaceutical in a range of salt concentrations, pHs, excipients, and pharmaceutical concentrations must be prepared and tested to find interactions between the pharmaceutical and excipients or interactions between excipients that affect the pharmaceutical's solubility. For example, a particular concern with frozen or freeze-dried formulations is premature precipitation during the freezing of the pharmaceutical preparation. While some general rules exist, the effect of individual excipient and excipient combinations on the physical and chemical properties of the pharmaceutical is not easily predicted.

There are over 3,000 familiar excipients to choose from when designing pharmaceutical formulations, each having different degrees and types of interactions with each other and with the pharmaceutical. Because of the many variables involved, industry does not have the time or resources to identify, measure, or exploit interactions between excipients and pharmaceuticals and thus cannot provide optimized pharmaceutical formulations tailored to the particular pharmaceutical. Such work would require testing hundreds to thousands of samples a day. Assuming three hundred substances are to be tested for efficacy as excipients in a pharmaceutical formulation, even with no variations in concentrations and no physical or chemical property variations, the number of possible combinations is enormous: when two of the substances are selected, there are 44,850 possible combinations; for three components there are 4,455,100 combinations; and for four components, there are 330,791,175 possible combinations. The complexity is increased when the relative ratio of each component is considered along with the effect of each component on freeze-drying of the formulation.

Not surprisingly, technologies that can test many pharmaceutical-excipient combinations suitable for further optimized processing via freeze-drying and or just freezing are not known. Today, since it is more cost effective, most pharmaceuticals are distributed and administered in standard, un-optimized formulations, see e.g., Allen's Compounded Formulations: U.S. Pharmacists Collection 1995 to 1998, ed. Lloyd Allen. Present day pharmaceutical formulation research and development relies on a select few excipients and retro-fits the active ingredients into well-known oral or parenteral formulation systems, a strategy that is further compounded by the need to evaluate the role of proposed excipients in freeze-drying.

The need to provide optimized formulations is not limited to formulations wherein the active component is a pharmaceutical. Similar problems are encountered for administering dietary supplements, alternative medicines, nutraceuticals, sensory compounds, agrochemicals, food products, and consumer and industrial product formulations. For example, similar to a pharmaceutical formulation, a vitamin formulation can be characterized by poor stability, solubility, bioavailability, taste, or smell.

Moreover, preserving the structure and/or function of biological specimens such as tissues and cells, whether live as in sperm, eggs, and embryos, or dead as in specimens, including those made by synthetic means, e.g., self-assembling systems, for observation or further processing to elucidate underlying structure, and even preserving the texture of food stuff presents problems that are well-suited for application of freezing and/or freeze-drying provided the appropriate conditions can be readily identified.

Freezing Chemical Compositions and Specimens of Interest

The freezing of a preparation containing water is typically accompanied by crystallization of a significant fraction of water in the form of ice. The process of crystallization is one of ordering. During this process, the ice crystals being formed grow rapidly with a larger volume than that of the liquid water contained therein. Such crystal growth often damages structures, for instance, biological structures such as cell walls, sub-cellular compartments, filaments and the like. Moreover, the liquid phase excluded from the ice crystals becomes increasingly concentrated with a greater likelihood of denaturation, precipitation, or modification of solutes. A goal for freezing is to reduce the extent of ice formation, since it excludes solutes, and promote sufficiently rapid freezing to minimize the risk of precipitation or denaturation of solutes. Notably, water is not the only component that may form crystals during freezing. The solutes may themselves crystallize, including crystals that contain water, either in stoichiometric association or in inclusions.

The term precipitation is usually reserved for formation of amorphous substances that have no symmetry or ordering and cannot be defined by habits or as polymorphs. Bio-precipitation processes can result in organic deposits in the biological specimens. Both crystallization and precipitation result from the inability of a solution (e.g., body or intra-cellular fluid) to fully dissolve the substance and can be induced by changing the state of the system in some way. Common parameters that can promote or discourage precipitation or crystallization include pH; temperature; concentration; and the presence or absence of inhibitors or impurities.

A process akin to crystallization that is typically limited to formation of substances displaying local order is that of deposition or polymerization of proteins and other molecules resulting in deposits and other aggregates. Such deposits or polymers may not be readily reversible, and hence formulation of such can compromise reconstitution and/or viability of a formulation or possibly introduce artifacts therein.

Important processes in crystallization and precipitation are nucleation, growth kinetics, interfacial phenomena, agglomeration, and breakage. Nucleation results when the phase-transition energy barrier is overcome, thereby allowing a particle to form from a supersaturated solution. Agglomeration is the formation of larger particles through two or more particles (e.g., crystals) sticking together. Supersaturation, defined as the deviation from thermodynamic equilibrium, is the thermodynamic driving force for both nucleation and growth.

Furthermore, the same solvent or solute compound can crystallize in different external shapes, termed habit, depending on, amongst others, the composition of the crystallizing medium. Such information is important because the crystal habit may influence the rate of sublimation and the associated primary and secondary drying times. Although crystal habits have the same internal structure and thus have identical single crystal- and powder-diffraction patterns, they can still exhibit different pharmaceutical properties (Haleblian, J.,“Characterization of Habits and Crystalline Modification of Solids and Their Pharmaceutical Applications”, J. Pharm. Sci., 64:1269, 1975). Crystal size and shape also have a great effect on the ease of removal of solvent and the extent of damage to structural elements of the specimen. Thus discovering conditions or pharmaceuticals to control or modulate crystal habit are needed for optimizing freeze-drying or freezing preparations where crystallization takes place.

Additionally, the same compound can crystallize as more than one distinct crystalline species (e.g., polymorphs having a different internal structure and physical properties) that may differ in ease with which sublimation may be carried out. Thus, determining conditions, compounds, or compositions that prevent shift to an unfavorable polymorph or promote shift to a more favorable polymorph are desirable.

Freezing of Biological Specimens

In contrast to chemical compositions, freezing of biologics present additional considerations based on whether structural integrity is of interest and/or viability is desired following thawing of a biological sample. Freeze-drying, including partial drying to remove water, is used to preserve biologics for later reconstitution into viable cells, tissue, organs, or even organism. In this aspect, freezing is often a critical step since damage resulting in compromised viability occurs during freezing. In many applications, the precise manner of freezing is of interest with parameters including the rate of cooling and the rate of thawing. Moreover, for many applications only the freezing (and the thawing) conditions are of interest with the subsequent drying via sublimation omitted.

Structural Investigations

Structural investigation of biologics is often performed by examination via electron microscopy. To this end, thin sections of a biological sample are placed on a metal grid, typically with additional treatment to ensure that the structure of interest is visible and stable. Artifacts are commonly encountered due to sample handling and the need exists to remove water prior to placing the sample in vacuum in the path of the electron beam. Thus, structures actually visualized may not reflect actual structures since drying subjects them to distortions due to surface tension.

Freeze-drying of biological specimens is a means for avoiding distortions of structure associated with the removal of water. For example, specimen samples can be prepared by using adsorption to secure them to a grid, and just before the liquid film dries, the grid is plunged into liquid nitrogen (or, better yet, into a cryoprotectant such as isopentane or ethane cooled to liquid nitrogen temperature). The frozen material is held at low temperature (approximately −100 to −80 degrees C.) in a vacuum until the water leaves by sublimation. The specimen is relatively rigid in the frozen state, thus reducing or eliminating forces due to surface tension.

Preserving Viable Biological Samples

In addition, biologics frozen in a viable state may be processed by techniques other than sublimation of frozen water both before and after freezing to control the formation of ice crystals or precipitates or complexes that compromise or promote viability. Some common treatments include using polyethylene glycol (PEG), dimethyl sulfoxide (DMSO), salts, alcohols, solvents, and the like. Some example biologics of interest for freezing are embryos, human embryos, human and animal sperm and/or eggs, organs, bacteria and yeast, and even whole organisms.

Some of the techniques directed at preserving function of biologics include identifying conditions for promoting vitrification, an amorphous glassy state, to avoid ice formation. Song et al. report in “Vitreous cryopreservation maintains the function of vascular grafts,” volume 18 (2002) of Nature Biotechnology on pages 296-299 that one approach to cryopreservation has been to discover conditions under which the lowest amount of a cryoprotective agent can still result in glass formation. Such conditions have to include additional variables to optimize the ionic composition and selection of a less toxic cryoprotective agent. Song et al. employed rings of vascular tissue to explore various combinations of freezing conditions with vitrification corresponding to relatively successful freezing as evaluated by the response of the thawed vascular rings to a variety of agonists and antagonists.

Attempts to identify conditions for preservation of function in frozen samples have been ongoing. The rather limited success, in part due to the large number of variables that need to be studied, is discussed in various reports. For instance, Gosden et al. describe the use of tissue slices to investigate cryopreservation of an organ in “Restoration of fertility to oophrectomized sheep by ovarian autografts stored at −90° C.” in volume 9 (1994) of Human Reproduction on pages 597-603. Various attempts to preserve functional kidneys are reported, for instance, by Jacobsen et al. in “Effect of Cooling and Warming Rate on Glycerolized Rabbit Kidneys” in volume 21 (1984) of Cryobiology on pages 637-653. In short, determining better freezing and thawing conditions for preservation of viable biological materials is currently significant and difficult due to the large number of variables affecting successful freezing and thawing.

Sublimation

Sublimation, the foundation of freeze-drying, is the process of phase change from a solid phase to vapor without an intervening liquid phase. Sublimation of a substance takes place under temperature and pressure conditions below the triple point in a phase diagram for the substance. The vapor phase and solid phase are in equilibrium under such conditions and lowering of the vapor pressure or raising of the temperature results in faster vaporization. Thus, providing a sink for the vapor phase, typically in the form of a pump for removing the vapor and/or a condenser, makes the process continuous. As might be expected, a source of heat is required to maintain the rate of sublimation since sublimation results in removing heat in the form of the latent heat of sublimation. Heat, typically sufficient to maintain the temperature of the solid phase, may be provided by conduction, convection, or radiation. Moreover, radiative heat transfer may result in heating just the surface of the solidified substance or provide for more extensive volumetric heating, e.g., by use of microwaves. See, Use of Volumetric Heating to Improve Heat Transfer During Vial Freeze-Drying, Ph.D. Dissertation of James B. Dolan submitted in September 1998 to the Faculty of the Virginia Polytechnic Institute and State University, which is incorporated by reference herein in its entirety.

Sublimation is particularly suitable for drying heat sensitive products that cannot be dried by other methods due to the high temperatures necessarily associated with alternative methods. Some example products preserved by sublimation are blood products, bone, skin, and labile biochemicals. Not surprisingly, freeze-drying results in improving the quality of the products compared to other methods of processing for preservation of products. Although the basic concepts of freeze-drying are known, the details are not determinable readily enough to circumvent costs imposed by the need for extensive experimentation for optimizing the process.

For carrying out sublimation of an aqueous preparation, typically the temperature is reduced below the freezing point of water and the pressure is reduced to below the saturated vapor pressure corresponding to the temperature of the frozen preparation. This results in progressive removal of water via sublimation leaving behind a structure formed by the excluded solutes as the water freezes into ice crystals. Preferably, this structure is very porous and is usually readily reconstituted. Moreover, the product comprising this dried structure is stable at both shelf (e.g., room temperature) and freezing temperatures provided the conditions during the freeze-drying avoided the collapse temperature. In the case of products having a supporting cellular structure, such as fruits, and biologics, freeze-drying can be carried out at higher temperatures than products initially in a solution state such as coffee (for making freeze-dried instant coffee). Of course, additional temperature limits may be dictated, for instance, by the range of temperatures acceptable for an active ingredient in a pharmaceutical preparation.

There are many more considerations in improving or devising a process for freezing or freeze-drying a product. Some example factors include the freezing temperature, the freezing rate, the degree of undercooling, the collapse temperature, the glass transition temperature, solvent held in an amorphous form or as residual moisture, annealing, the propensity to precipitate or polymerize, and the like.

Freezing Temperature

This is the temperature at which a liquid changes phase to become a solid, with a concomitant release of latent heat of melting. The temperature at which a solvent freezes may vary depending on, for instance, the composition of the solvent and the probability of nucleation of the solid phase. As is well known, addition of a salt to water lowers the freezing point. Moreover, as water freezes, salt is largely excluded from the ice crystals resulting in a higher salt concentration in the liquid phase, which further lowers the freezing point for the remaining solution. Anti-freezing agents, including naturally occurring anti-freeze proteins, may also act by interfering with the formation or growth of ice crystals and thus lower the freezing temperature as well.

Freezing Rate

The rate of freezing can be an important parameter for preparing a suitable solid phase material for preserving a specimen or carrying out freeze-drying. Typically, the size, nature and extent of precipitation of solutes is affected by the freezing rate. Rapid freezing may result in an amorphous mass with little crystalline structure or only relatively small crystals as well as less precipitation.

Directional freezing is also possible by nucleating one side of the sample to allow crystals to grow from that side. This results in more directional channels in the cake left behind upon subsequent sublimation with faster primary and secondary drying (described below).

Undercooling

Undercooling, also termed supercooling, is the phenomenon of bringing the temperature of a liquid below its equilibrium freezing point without crystallization. As an example highly pure water may remain in a liquid state as much as 40° C. below its freezing point due to the low rate of nucleation. The degree of undercooling is the difference between the temperature of a liquid and its equilibrium freezing temperature.

Freeze-Concentration

As previously mentioned, freezing results in the exclusion of a solute from the crystals of a solvent resulting in a more concentrated solution. This phenomenon is also termed freeze-concentration.

Effect of Chamber Pressure

Chamber pressure includes the pressure contributions from all vapor phase species although sublimation proceeds towards vapor phase if the solvent species partial pressure is below its saturated vapor pressure. When the chamber pressure is reduced below the saturated vapor pressure at a corresponding temperature, freeze-drying is promoted regardless of the partial pressure contribution due to other species. The chamber pressure also affects heat transport characteristics. This is significant since transition of the frozen solvent into the vapor phase requires heat. Typically, heat also flows to the frozen sample undergoing sublimation by convection, a process that is adversely affected by a reduction in the chamber pressure. A lower chamber pressure typically increases the thermal contact resistance to conductive heat flow.

Formulation Microstructure

Formulation microstructure refers to the solute left behind following freeze-drying (e.g., after a sublimation front passes through a region leaving behind the residue). The microstructure of the residue, the formulation microstructure, should desirably be porous to facilitate reconstitution by providing a large surface area to an added solvent. The formulation microstructure may be unstable at the temperatures required for economical storage and may require additional processing to stabilize (e.g., by additional drying to remove residual solvent or selecting suitable excipients).

Thermal Gradient

During sublimation all parts of the frozen phase have to be maintained at a temperature that is lower than prescribed limits while ensuring heat flow to the interface between the solid and the vapor phase. These limits are often, but not necessarily, determined by the need to avoid a collapse of the freeze-dried material left behind by the solid/vapor interface as it moves further into the frozen solid phase. The heat flow, on the other hand, depends on the thermal gradient between the walls and the solid/vapor interface. In view of the aforementioned limits, often a high thermal gradient to increase the heat flow is not possible.

Moreover, controlling the temperature and the thermal gradient is complicated by the thermal capacitance of the frozen mass. As sublimation proceeds, the solid/vapor interface moves leaving behind a freeze-dried layer that provides additional resistance to removal of vapor, thus lowering the rate of sublimation based solvent removal.

Glass Transition

A glass is a solid lacking an ordered crystalline structure. Typically the formation of a glass does not require a nucleation event, Glass transition is not a phase transition in the sense of a solid to liquid or vapor transition. Instead, it is characterized by an increase in viscosity with a rapid increase in viscosity near the glass transition temperature as temperature is reduced. The glass transition temperature is usually the midpoint of this transition region exhibiting a rapid increase in viscosity.

In the context of freeze-drying, the glass transition temperature typically refers to the formation of a glass by the residue left following the removal of solvent via primary drying. The glass transition temperature of this material depends on the extent of residual solvent. Typically, the glass transition temperature increases as the residual moisture decreases with the end of secondary drying characterized by an acceptably high glass transition temperature.

Collapse Temperature

As noted previously, viscosity increases rapidly at a temperature below the glass transition temperature. If the glass transition temperature is lower than the temperature at which a freeze-dried preparation is to be stored, then the freeze-dried preparation will exhibit insufficient viscosity to resist flow, which results in collapsing on itself. Such a collapse drastically reduces the surface area resulting in a substance that is difficult to reconstitute and possibly is mechanically damaged as well. It should be noted that the collapse mentioned herein is due to flow of the material and not parts of the freeze-dried material mechanically fracturing, e.g., due to handling, vibrations, and the like.

Continued drying reduces the residual solvent with concomitant increase in the glass transition temperature. A glass transition temperature that is equal to the temperature for storing or handling the freeze-dried preparation is termed the collapse temperature. Therefore, one of the objectives of freeze-drying is to ensure that the temperature of the sample never exceeds the collapse temperature for a significant duration, if at all. Thus, for a pharmaceutical intended to be stored or exposed to room temperature in its freeze-dried state, it is important to ensure that the glass transition temperature of the freeze-dried preparation is higher than room temperature.

Put another way, for a freeze-dried preparation, the collapse temperature is the temperature at which the preparation begins to flow, i.e., collapse. Therefore, the temperature of the sample during the freeze-drying process should not exceed the collapse temperature. In the pharmaceutical context, collapse refers to the degradation of the structure of the dried product as the sublimation interface passes through it during primary drying. As might be expected, the glass transition temperature, and hence the collapse temperature, also depends on the composition of the solutes left behind. Thus, determining the collapse temperature for a pharmaceutical preparation also indicates the limits on the rate at which sublimation can be carried out safely with a high thermal gradient.

Maximum Storage Temperature

The maximum storage temperature, typically lower than the collapse temperature/glass transition temperature, is the temperature at which the extent of flow in the freeze-dried preparation in the course of storage is acceptable.

The Residual Moisture/Solvent

The residual moisture/solvent is the water or solvent in general, left behind following primary drying. Alternatively, it maybe considered to be the solvent that is not sequestered in crystals upon freezing a liquid.

Annealing

The size of solvent crystals can be increased by freezing a sample (e.g., by supercooling) followed by warming the sample to within a few degrees below the melting point of the solvent. Preferably, this temperature is no more than ten degrees below the melting point, more preferably this temperature is no more than five degrees below the melting point, even more preferably this temperature is no more than two degrees below the melting point and most preferably, this temperature is no more than one degree below the melting point of the solvent. By keeping the sample frozen in this manner, the solvent crystals grow and rearrange themselves. Upon sublimation they leave behind larger and less tortuous paths, with concomitant increase in the rate of sublimation due to lower impedance for vapor transfer through the residue.

Primary Drying

Removal of crystallized solvent from a frozen preparation via sublimation is carried out in the primary drying stage. Primary drying is typically the longest time period required for freeze-drying. During primary drying a substantial fraction of solvent crystals in the frozen sample are removed via sublimation. It may be considered to have more than one sub-stage, e.g., for removal of solvent forming more than one crystalline form that exhibit different drying rates.

Secondary Drying

In addition to crystalline solvent, additional solvent molecules are associated with a frozen preparation. These may assume an amorphous form or be part of the solute as water of crystallization and the like. Removal of such solvent molecules is limited by diffusion through the solid phase to the surface and is effected by desorption.

Removal of amorphous or sequestered (e.g., as water of crystallization) solvent from a frozen preparation via sublimation is carried out in the secondary drying stage. Chemical means of removing the solvent vapor may be required since it is not always possible to sufficiently reduce the chamber pressure. This follows from the fact that some of the solvent removed during secondary drying is held more tightly due to interactions with the residue. It may be considered to have more than one sub-stage (e.g., for removal of solvent being held with varying degrees of affinity resulting in different drying rates).

At present, industry does not have the time or resources to test hundreds of thousands of combinations to find the right conditions, compounds, or compositions adverse to undesired physical-state changes during freezing and freeze-drying in a time efficient and cost effective manner. To remedy these deficiencies, methods for rapid screening of conditions, compounds, or compositions of thousands to hundreds of thousands of samples per day, cost effectively, are needed. The invention disclosed herein addresses the issues discussed above.

### SUMMARY OF THE INVENTION

Methods, systems, and devices are disclosed for practical and cost-effective rapid production and screening of hundreds, thousands, or hundreds of thousands of samples per day. This provides an extremely powerful tool for the rapid and systematic analysis, optimization, selection, or discovery of conditions and compositions suitable for freezing and/or freeze-drying in a cost-effective manner.

The disclosed methods, systems, and devices encompass optimization, selection, or discovery of compounds or compositions exhibiting a high collapse temperature to help stabilize during storage the formulation microstructure of a freeze-dried product. The invention further encompasses the use of such compounds or compositions following reconstitution for treating, or preventing a disease itself, the cause of the disease, or the symptoms of the disease.

The invention further encompasses a method for the discovery of physiological conditions (e.g., pH, salt concentration, protein concentration, etc.) that inhibit or prevent damage to biologics due to the crystallization of water, precipitation, formation, or deposition of inorganic or organic substances upon freezing. In particular, these physiological conditions prevent damage to biological structures, prevent the generation of artifacts, and/or prevent a loss of viability of biologics due to freezing.

The invention further encompasses methods to discover compounds, compositions, or physiological conditions that prevent or inhibit unfavorable crystallization or precipitation of pharmaceuticals upon freezing in the course of freeze-drying.

In one embodiment, the invention comprises high throughput screening of arrays, each array having at least 24, 48, 72, 96, 384, or 1536 samples, to identify conditions, compounds, or compositions exhibiting a high collapse temperature to help stabilize the formulation microstructure during storage as a freeze-dried product. The invention further encompasses the use of identified compounds or compositions following reconstitution for treating or preventing a disease itself, the cause of the disease, or the symptoms of the disease.

In another embodiment, the invention concerns a method of preparing and screening an array of at least 24, 96, 384, or 1536 samples to identify conditions, compounds, or compositions that inhibit or prevent damage to biologics due to crystallization of water, precipitation, formation, or deposition of inorganic or organic substances upon freezing. In particular, these physiological conditions prevent damage to biological structures, and/or avoid artifacts, and/or reduce the loss of viability of biologics upon freezing. The method includes adding a freezing solution to each of the samples to freeze them by cooling them at a pre-determined cooling rate.

In still another embodiment, the invention relates to a method for discovering compounds or compositions suitable for freezing comprising preparing an array of samples, each sample comprising a plurality of components, and freezing the samples followed by sublimation of the solvent by adjusting the pressure such that a partial pressure due to the solvent is lower than the saturated vapor pressure for the solvent. Subsequent or concurrent analysis of the samples allows selection of samples exhibiting desired properties or characteristics.

In yet another embodiment, the invention comprises a plate assembly having a plurality of optically clear layers that form a plurality of chambers with ports such that each chamber is accessible for adding a sample formulation and application of a predetermined vacuum to the chamber. A pressure and temperature controlled chamber suitable for use as a stage in a microscope receives the assembled stack of the plurality of optically clear plates and provides ports for vacuum, coolant, and electrical and electronic connections. A cooling system having plates with integral cooling channels provides a main cooling source with a thermoelectric fine temperature control system and a system of fins and plates to conduct cooling to the assembled stack of the plurality of optically clear plates. This arrangement enables examination and evaluation of samples while freezing and freeze-drying under a microscope.

These and other features, aspects, and advantages of the invention will become better understood with reference to the following detailed description, examples, and appended claims.

### DESCRIPTION OF THE FIGURES

FIG. 1 illustrates a lyophilization plate for a freeze-drying microscope stage

FIG. 2 shows a plurality of stacked optically clear layers in a lyophilization plate

FIG. 3 illustrates an exemplary lyophilization chamber in an exploded view

FIG. 4 illustrates another exemplary lyophilization chamber in an exploded view

FIG. 5 illustrates a layer providing a plate containing holes or chambers for sample well(s) in an assembled lyophilization plate

FIG. 6 illustrates a side view of a cooling arrangement in a lyophilization chamber

FIG. 7 illustrates the deployment of the lyophilization chamber as a stage in a microscope

FIG. 8 shows one set of dimensions for a lyophilization plate

FIG. 9 shows dimensions for individual wells for sample placement regions

FIG. 10 shows a side view of a lyophilization plate with a lyophilization plate bottom layer, a lyophilization plate middle layer, and a lyophilization plate top layer

### DETAILED DESCRIPTION OF THE INVENTION

As an alternate approach to traditional methods for discovering freeze-dried pharmaceutical formulations, or frozen biological specimens or biologics exhibiting superior preservation of structure and/or viability, applicants have developed practical and cost-effective methods for high throughput production and screening of hundreds, thousands, or hundreds of thousands of samples per day. These methods are useful to systematically optimize, select, and discover compounds, compositions, or conditions for freeze-drying. For example, these methods are useful to optimize, select, and discover compounds, compositions, or conditions that prevent or inhibit undesirable crystallization, precipitation, formation, or deposition of inorganic and organic substances in response to freezing.

In the preferred embodiment, the samples are prepared in a grid or array (i.e., an ordered set of components) such as a 24, 36, 48, 72, 96, 384, or 1536, or other standard arrays, e.g., as wells in a plate. In addition, arrays suitable for processing at least 200, 500, 1000, 5000, 10,000, or 100,000 samples can be used. Each sample in the array comprises a formulation containing one or more excipients suitable for freezing or freeze-drying. In addition, a sample may alternatively include biological material, possibly treated to reduce the water content or remove waxy coats/deposits and the like, together with a medium suitable for freezing. In some instances, biological material, such as cells and tissues, may be cultured in multi-well plates followed by optional washing and change of medium prior to freezing.

The disclosed methods, systems, and devices encompass optimization, selection, or discovery of compounds or compositions exhibiting a high collapse temperature to help stabilize during storage the formulation microstructure of a freeze-dried product. The invention further encompasses the use of such compounds or compositions following reconstitution for treating, or preventing a disease itself, the cause of the disease, or the symptoms of the disease. The invention also encompasses methods to discover compounds, compositions, or physiological conditions that prevent or inhibit unfavorable crystallization or precipitation of pharmaceuticals upon freezing in the course of freeze-drying.

The invention also comprises a layered plate assembly with a plurality of optically clear layers forming a plurality of chambers such that each chamber is accessible for adding a sample formulation and application of a predetermined vacuum to the chamber via one or more ports. The plate assembly may be placed in a pressure and temperature controlled chamber and used as a stage in a microscope to examine samples in a selected chamber during freezing, freeze-drying, and/or thawing/warming. A thermoelectric fine temperature control system in combination with a cooling system having plates with integral cooling channels provides accurate control of the cooling rate.

The array or selected samples therein can be subjected to processing parameters. Examples of processing parameters that can be varied include temperature, temperature gradient, time, pressure, the identity or the amount of excipient, crystallinity, density, and the like.

After processing, each sample in the processed array may be screened to determine changes in physical state, particularly changes in the microstructure upon freezing, by techniques such as phase contrast microscopy, transmission microscopy, confocal microscopy, 2-photon microscopy, or a CCD camera. But a simple visual analysis can also be conducted including photographic analysis, optionally, coupled with software for image processing.

Definitions

High Throughput:

High throughput refers to the handling of at least 100, 1000, or 10,000 samples. This handling is preferably during the course of one month, one week, three days, or one day.

Array:

As used herein, the term “array” means a plurality of samples, preferably, at least 24 samples. Each sample comprises a formulation being tested for stability under freeze-thaw cycles and subsequent freeze-drying although in some instances the freeze-drying testing may be omitted. This preceding description should not be interpreted to exclude negative controls. Each sample can have different components or concentrations of components. An array can comprise 24, 36, 48, 72, 96, 384, 1536, or more samples, preferably 1000 or more samples, more preferably, 10,000 or more samples. Moreover, an array may comprise one or more groups of samples also known as sub-arrays.

Precipitated or Deposited Substance in the Course of Freezing:

As used herein, the term “precipitated or deposited substance in the course of freezing” means any solid, semisolid, paste, gel, or plaque formed in the course of freezing a sample of interest. Examples of precipitated or deposited substances in the course of freezing include, but are not limited to, salts and compositions thereof; protein precipitates and deposits; and combinations thereof and the like. In addition, exceeding the collapse temperature during freeze-drying may result in the formation of precipitated or deposited substance.

Sample:

As used herein, the term “sample” typically means a formulation containing one or more excipients suitable for freezing/freeze-drying. In addition, a sample may alternatively include biological material, possibly treated to reduce the water content or remove waxy coats/deposits and the like, together with a medium suitable for freezing. In some instances, biological material, such as cells and tissues, may be cultured in multi-well plates followed by optional washing and change of medium prior to freezing. Preferably, the sample has a total volume of about 1 microliter, or about 5 microliters to about 500 microliters, or about 10 microliters to about 200 microliters.

Component:

As used herein, the term “component” means any substance that is combined, mixed, or processed in a sample. A single component can exist in one or more physical states. Examples of suitable components include, but are not limited to, DMSO, alcohols, acetone, salts, proteins, and carbohydrates for modulating solvent/medium crystallization, precipitation, formation of inorganic and organic deposits; small molecules (i.e., molecules having a molecular weight of less than about 1000 g/mol); large molecules (i.e., molecules having a molecular weight of greater than about 1000 g/mol), such as oligonucleotides, proteins, and peptides; hormones; steroids; matrix and connective tissue, such as cartilage and collagen; biological-membrane extracts; chelating agents, such as EDTA; excipients; organic solvents; water; salts; acids; bases; gases; and stabilizers, such as antioxidants.

Processing Parameters:

As used herein, the term “processing parameters,” also referred to as conditions for freeze-drying, means the physical or chemical conditions for carrying out the freezing or freeze-drying of a sample of interest. Processing parameters include, but are not limited to, adjustments in time of incubation, temperature, solvent vapor pressure, total pressure, pH, and chemical environment. Processing also includes adjusting the concentration of components, adding various additional components, or adjusting the composition or amounts.

Sub-arrays or even individual samples within an array can be subjected to processing parameters that are different from the processing parameters to which other sub-arrays or samples, within the same array, are subjected. Processing parameters will differ between sub-arrays or samples when they are intentionally varied to induce a measurable change in the sample's properties. Thus, according to the invention, minor variations, such as those introduced by slight adjustment errors, are not considered intentionally varied.

Physical State:

Physical state includes presence or absence of non-stoichiometric solvates and hydrates including inclusions or clathrates, that is, where a solvent or water is trapped at random intervals within the crystal matrix, for example, in channels. Of course, such water is also a part of the solute glass left behind by the freezing out of significant amount of water as ice crystals.

A stoichiometric solvate or hydrate is where a crystal matrix includes a solvent or water at specific sites in a specific ratio. That is, the solvent or water molecule is part of the crystal matrix in a defined arrangement. Additionally, the physical state of a crystal matrix can change by removing a co-adduct, originally present in the crystal matrix. For example, if a solvent or water is removed from a solvate or a hydrate, a hole is formed within the crystal matrix, thereby forming a new physical state (e.g., during secondary drying). Such physical states are referred to herein as dehydrated hydrates or desolvated solvates.

The crystal habit is the description of the outer appearance of an individual crystal, for example, a crystal may have a cubic, tetragonal, orthorhombic, monoclinic, triclinic, rhomboidal, or hexagonal shape. The internal structure of a crystal refers to the crystalline form or polymorphism. A given compound may exist as different polymorphs, that is, distinct crystalline species. In general, different polymorphs of a given compound are as different in structure and properties as the crystals of two different compounds. Solubility, melting point, latent heat of sublimation, density, hardness, crystal shape, optical and electrical properties, vapor pressure, and stability, etc. may vary with the polymorphic form.

Treatment:

Treatment includes curative, palliative, and/or preventive administration of a substance for curing, managing, or avoiding a disease state. Thus, a freeze-dried substance is administered directly or following re-suspension in a suitable medium to a subject in the course of a treatment. This administration may be oral, intravenous, in conjunction with a surgical procedure, as a suppository, spray, liquid, and/or powder.

The array technology described herein is an approach that can be used to generate large numbers (greater than 10, 50, 100, or 1000,) of parallel small scale samples.

System Design for Preparing and Screening Arrays:

The basic requirements for array and sample preparation and screening thereof are: (1) a distribution mechanism to add components and the medium to separate sites, for example, on an sub-array plate having sample wells or sample tubes. Preferably, the distribution mechanism is automated and controlled by computer software and can vary at least one addition variable (e.g., the identity of the component(s) and/or the component concentration). Examples of such material handling technologies and robotics, well known to those skilled in the art, include automated liquid distribution mechanisms, such as the Tecan Genesis, from Tecan-US, RTP, North Carolina. Of course, if desired, individual components can be placed at the appropriate sample site manually.

For preparing, processing, and screening an array, an optional set of steps comprise selecting the component sources, preferably, at one or more concentrations, adding the components to a plurality of sample sites, such as sample wells or sample tubes on a sample plate to give an array or sub-array of samples. A preferred sample plate is the lyophilization plate described herein. The data so collected are stored for subsequent data analysis, preferably, by a computer.

Preferably, the automated distribution mechanism used in accordance with the invention can distribute or add components in the form of liquids, solids, semi-solids, gels, foams, pastes, ointments, suspensions, or emulsions. Automated liquid distribution mechanisms are well known and commercially available, such as the Tecan Genesis, from Tecan-US, RTP, North Carolina. These may be modified to accurately dispense viscous fluids. Moreover, these may be supplemented by solid dispensing mechanisms, including mechanisms for dispensing small amounts of solids such as less than 10.0 mg, 1.0 mg, 100 micrograms, or 10 micrograms.

After dispensing is complete the plates can be sealed and placed in a microscope stage providing control over temperature and pressure while enabling visual examination of the samples during and after cooling, freezing, thawing, re-freezing, and freeze-drying. The visual inspection of each sample enables estimation of collapse temperature, glass transition temperature, formulation microstructure including porosity, distribution of crystal sizes, and the like. Many parameters and conditions of interest are described next in a non-exhaustive list:

Temperature:

Different temperatures can be used during the freezing, primary drying and secondary drying of samples in an array. Typically, several distinct temperatures are tested for freezing. Temperature can be controlled in either a static or dynamic manner. Static temperature means that a set incubation temperature is used throughout the solid formation process. Alternatively, a temperature gradient can be employed. For example, the temperature is decreased or raised at a constant rate throughout the solid formation.

Time:

Samples can be incubated for various lengths of time. Since physical-state changes, particularly flow of a glass and drying even at a lowered temperature, can occur as a function of time, it is advantageous to examine arrays over a range of times.

PH:

The charge of the compound being precipitated or crystallized can influence freezing of the sample. To this end, the pH can be modified by the addition of inorganic and organic acids and bases, additional crystallization additives such as small molecules, macromolecules, and solvents.

Solvent Composition:

The use of different solvents or mixtures of solvents can inhibit or promote physical-state changes and influence the type of physical state change during freezing, and freeze-drying. Solvents may influence and direct the formation of precipitates and solids through electrostatic properties, charge distribution, molecular shape and flexibility, and pH. Preferred solvents are solvents accepted for use in drug manufacture and mixtures thereof Acceptable solvents include, but are not limited to, aqueous-based solvents such as water, aqueous acids, bases, salts, and buffers or mixtures thereof and organic solvents, such as protic, aprotic, polar or non-polar organic solvents.

Microscopy-Assisted Processing and Examination:

Microscopy-assisted processing and examination involves observation of crystals, residue, and physical-state changes during freezing and freeze-drying under a microscope. In one embodiment, the array can be processed at a temperature (T1) at which the solids are in solution. The samples are then cooled, to a lower temperature (T2) that is sufficient for freezing or initiating freezing. The presence of solids, and forms thereof, may be optionally determined, preferably by visual examination aided by microscopy. The several microscopy-based techniques that may be employed for microscopic examination of samples in the various embodiments of the invention are described next in a non-exhaustive manner:

Transmission Microscopy:

In transmission microscopy, light is passed through a specimen prior to formation of a magnified image. Transmission microscopy, as employed in the invention, includes techniques for improving resolution at high magnifications, for instance magnifications in excess of about 400×.

Confocal Microscopy

Confocal techniques make possible suppression of out-of-focus light based noise. Briefly, the image is reconstructed from a, typically, three dimensional scan of a sample being observed, e.g., raster scanning, such that the light collected passes through a pinhole. Passage through the pinhole effectively blocks light from above the focal plane and below the focal plane. This technique is of particular value when non-specific fluorescence or scattering is a significant experimental limitation.

Two-Photon Confocal Microscopy:

Two-photon confocal microscopy is based on the two-photon effect, by which a chromophore is excited not by a single photon of visible light, but by two lower-energy (infrared) photons that are absorbed contemporaneously (on the order of femtoseconds). Fluorescence from the two-photon effect depends on the square of the incident light intensity. Because of this highly nonlinear (approximate fourth power) behavior, only those dye molecules very near the focus of the beam are excited resulting in reduced photobleaching, phototoxicity, or heating of the specimen during confocal imaging.

Phase-Contrast Microscopy:

Phase-contrast microscopy is used to obtain sufficient contrast between structures with similar transparency and no color by resolving structures based on their respective refractive indices.

CCD Camera:

A charge-coupled device (CCD) camera uses a small, rectangular piece of silicon, rather than a piece of film to receive incoming light. This solid-state electronic component is a micro-manufactured and segmented into individual light sensitive cells packed at high densities. In an important aspect, in addition to sensitivity, CCD cameras are also well suited for integration with automated processing of the image data.

Automated Image Processing:

Automated image processing of image data may be performed using visualization software, such as SPOTFIRE (commercially available from Spotfire, Inc., Cambridge, Mass.). The data, including image data, can be analyzed directly or processed through data mining algorithms so as to optimize the ability of scientific personnel to detect complex multi-dimensional interactions or any lack of interactions. Examples of suitable data-mining software include, but not limited to, SPOTFIRE; MATLAB (Mathworks, Natick, Mass.); STATISTICA (Statsoft, Tulsa, Okla.). All resulting analysis files may be stored on a central file server, i.e., a database, for access by traditional means known to those skilled in the art.

In another embodiment, so-called machine vision technology is used. Specifically, a high-speed CCD camera with an on-board signal processor captures images. This on-board processor is capable of rapidly processing the digital information contained in the images of the sample tubes or sample wells. Typically, images are generated for each location of the well such that the changes in the structure of the freeze-dried residue are evaluated at different times during the process of freeze-drying to detect conditions resulting in a low collapse temperature or a longer than expected drying time. Differences in these images due to differential rotation of the polarized light may indicate the presence of one or more newly formed frozen solvent/solute crystals or other component aggregates. For wells that contain such crystals, the vision system may determine the number of crystals in the well and/or a size distribution of crystals in a well to evaluate the freezing or freeze-drying process.

Microscope Stages for Freeze-Drying:

Several companies provide microscope stages suitable for studying freeze-drying of individual samples. Some example companies are Cybertek, Leica Microscopy & Scientific Instruments Group, Leica Microsystems Holdings GmbH, Oxford Instruments, Inc., and Scientific Research Div., United Products & Instruments, Inc. Traditional stages which employ Peltier-effect cooling and joule heating are also commercially available for studying freezing and freeze-drying of individual samples.

With regard to employing the Peltier-effect for controlling the temperature of a plurality of samples, a standard thermocycler used for PCR (polymerase chain reaction), such as those manufactured by MJ Research or PE Biosystems, can also be modified to accomplish the temperature control of a plurality of samples in a microscope stage.

Freeze-Drying of Biological Samples:

Special precautions are needed for the freeze-drying of microorganisms sensitive to desiccation, light, oxygen, osmotic pressure, surface tension and other factors.

Normally extensive experimentation is required to discover the conditions conducive to the freeze-drying process of a difficult to preserve (by freeze-drying) organism. The costs of alternative preservation strategies such as freezing or propagation are substantial while freeze-drying potentially allows storage at significantly higher temperatures, even as high as room temperature.

Many effective protective agents of utility in freeze-drying are known. Examples include skim milk, meso-inositol, honey, glutamate, and raffinose, for protecting against injuries resulting from freezing. Several anaerobic bacteria which are sensitive to aerobic freeze-drying, can successfully be frozen using activated charcoal (5% w/v) in the suspending media along with one or more additional protective agents.

Improving the Rate of Freeze-Drying a Formulation:

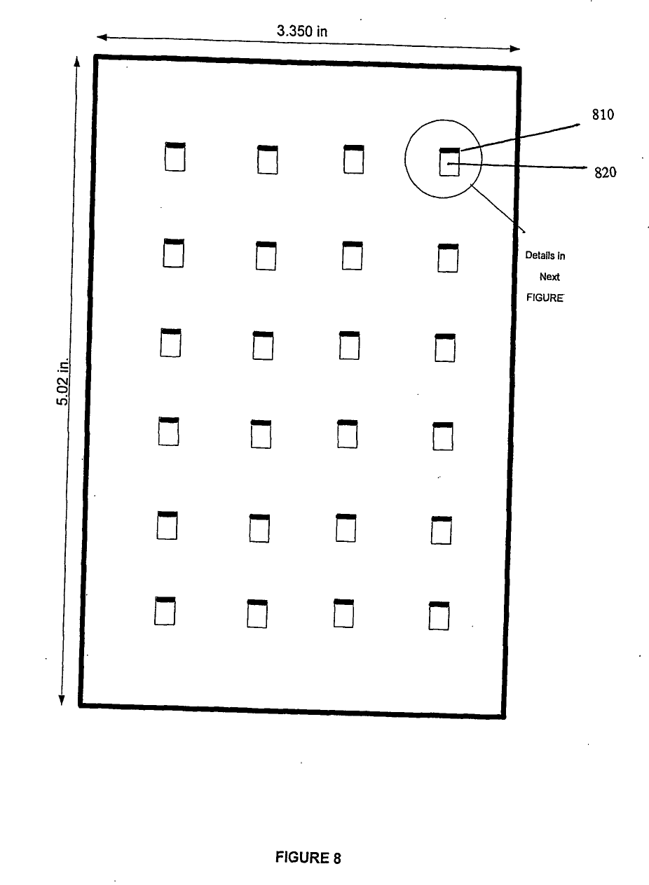

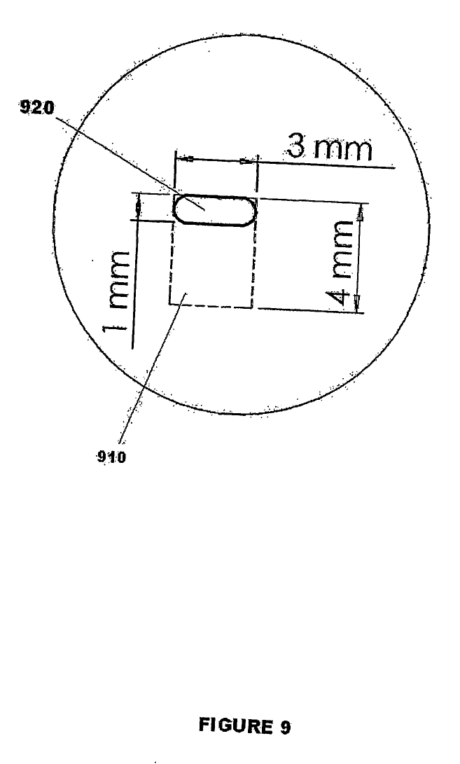

In contrast with trial and error strategies, the invention teaches systematic screening to improve the rate of freeze-drying along with developing more economic freeze-drying methods and apparatuses. The large number of variables playing a role in determining the freeze-drying process are examined by using one or more arrays of samples that allow examination of samples exposed to a range of temperature, pressure, time, and choice of compositions for determining desirable process parameters. Preferably the evacuation ports are aligned with an end of the sample placement regions (although completely within the sample placement region) to better view the sublimation front as it passes through the sample (see FIGS. 8 and 9).

An exemplary freeze-drying microscope stage that can accommodate several samples for microscopic examination of their structure, extent of freeze-drying, effect of cycles of freezing and thawing, enables screening an array of samples to identify processing parameters for freeze-drying. In addition, such a microscope stage is also useful for evaluating bio-viability of samples under different freezing conditions. Since a reliable prediction of desired conditions for freezing or freeze-drying a material of interest is not possible, in general, from first principles, a cost-effective screening of the effect of various component combinations and conditions is desired. This necessarily requires processing of a large number of samples and automation. The disclosed methods allow screening of a large number of samples and recording of such screening, if desired, by way of cameras and other devices. Various types of microscopes including confocal microscopes are suitable for the described freeze-drying stage.

To these ends the lyophilization plate has, for example, at least 5, 10, 24, 25, 50, 96, 100, 150, 200, 250, 500, 750, 1000, 2000 or 5,000 wells to allow reasonable sized batches for processing an array.

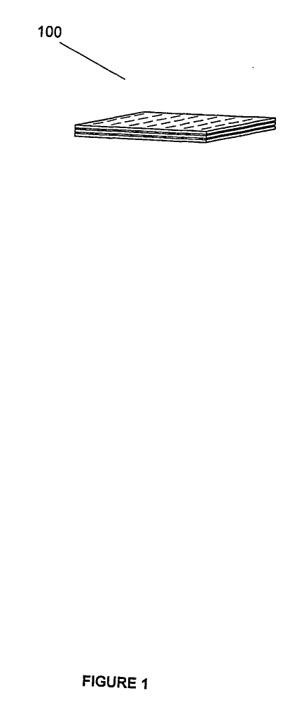

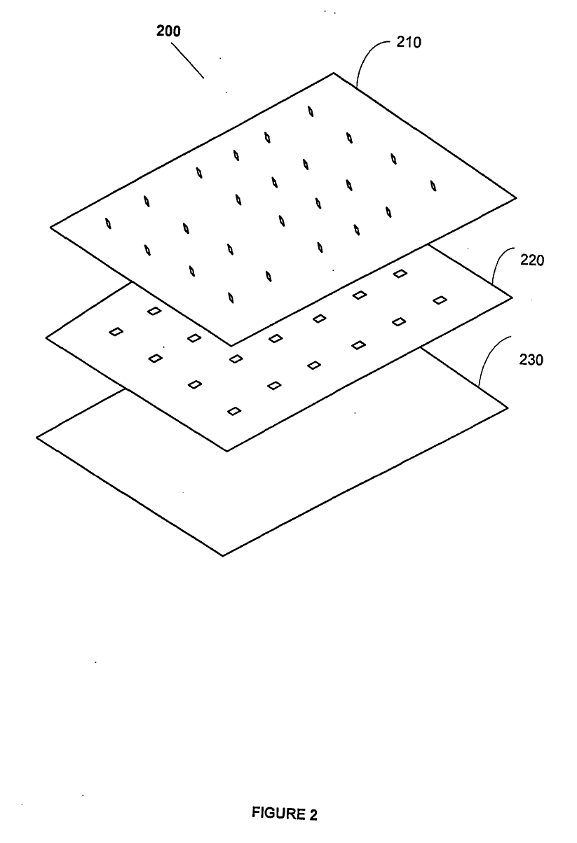

FIG. 1 illustrates lyophilization plate 100 for a freeze-drying microscope stage. Lyophilization plate 100 comprises a plurality of stacked optically clear-layers 200, with top layer 210, middle layer 220, and bottom layer 230, shown in FIG. 2. Top layer 210 has holes (vapor evacuation ports) that line up with the holes (sample placement regions) in the middle layer 220. The bottom layer 230 is generally solid. It should be noted that the depiction of three layers in the various figures herein is not intended to be limiting as to the scope of the invention. In one embodiment, for example, the lyophilization plate consists of 2 layers; a first top layer 210, and a bottom layer comprising wells or cavities to contain the samples. In the exemplary embodiment, a plurality of chambers are formed by stacking the optically clear-layers forming lyophilization plate 100. Advantageously, each of these chambers is observable to allow examination of a sample contained therein. The lyophilization plate is placed in a pressure and temperature controlled chamber having optically-clear windows for observing and illuminating samples placed in the lyophilization plate. Moreover, heating, cooling, and pressure controls connected to the freeze-drying microscope stage facilitate observing the array of samples under a variety of conditions.

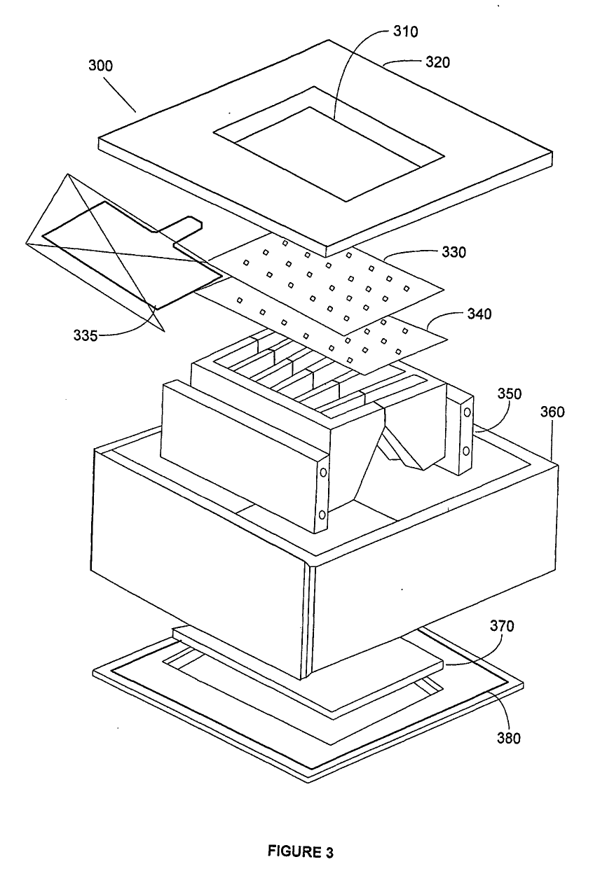

FIG. 3 illustrates an exemplary lyophilization chamber in an exploded view. Lyophilization chamber 300 has window 310 to facilitate observation of samples contained therein. Window 310 is associated with lyophilization chamber top 320 that rests on lyophilization chamber sides 360. Window 310 allows a view of lyophilization plate 330 resting on cooling transfer plate 340. Cooling transfer plate 340 may include temperature control or sensing elements such as one or more thermocouples and thermoelectric or other fine heating/cooling means. Cooling transfer plate 340 is also in contact with cooling assembly/fins 350 with coarse cooling provided by a coolant such as liquid nitrogen. Another window 370 allows trans-illumination in a microscope stage with lyophilization chamber bottom 380 providing a seal. In addition, FIG. 3 also depicts an alternative layer, laminated flexible heater and temperature sensor array 335. Laminated flexible heater and temperature sensor array 335 maybe attached to either lyophilization plate 330 or cooling transfer plate 340 or sandwiched between them to provide fine temperature control or sensing.

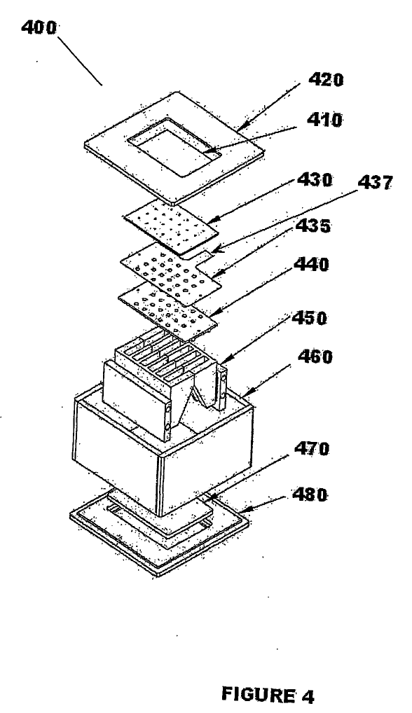

FIG. 4 illustrates another exemplary lyophilization chamber in an exploded view. Lyophilization chamber 400 has window 410 to facilitate observation of samples contained therein. Window 410 is associated with lyophilization chamber top 420 that rests on lyophilization chamber sides 460. Window 410 allows a view of lyophilization plate 430 resting on laminated flexible heater and temperature sensor array 435, connected to leads or input/output elements 437. Laminated flexible heater and temperature sensor array 435 is in contact with cooling transfer plate 440. Cooling transfer plate 440 may also include temperature control or sensing elements such as one or more thermocouples and thermoelectric or other fine heating/cooling means. Cooling transfer plate 440 is also in contact with cooling assembly/fins 450 with coarse cooling provided by a coolant such as liquid nitrogen. Another window 470 allows trans-illumination in a microscope stage with lyophilization chamber bottom 480 providing a seal. By way of illustrating various possible shapes for the wells, this embodiment has wells with a different shape than FIG. 3. The lyophilization chamber may optionally comprise other components such as outlets for electrodes and a vacuum source.

In a further embodiment, the apparatus of the present invention further comprises an optical device such as a digital camera or video recorder to view and store images of the samples.

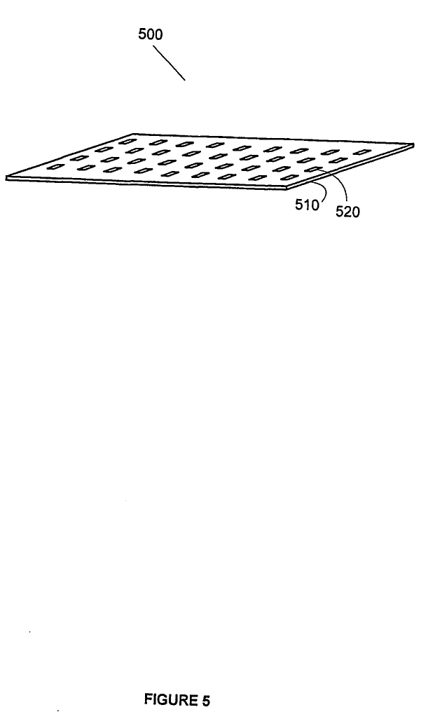

FIG. 5 illustrates layer 500 providing plate 510 containing holes or chambers for sample well(s) 520 in assembled lyophilization plate 100. The wells are sealed upon stacking of various layers. As is readily noted, there are various possible designs for generating and arranging wells. Such designs are intended to be included within the scope of the claimed invention. Advantageously, a regular pattern, although not necessarily a rectangular grid, allows use of automated processing of the wells rather easily.

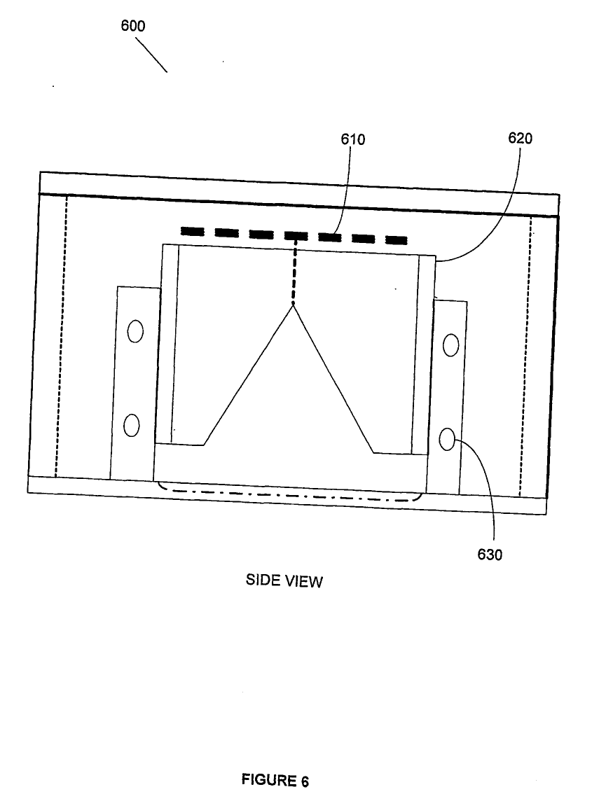

FIG. 6 illustrates a side view of cooling arrangement 600 in the described embodiment. Cooling transfer plate 610 is transparent to light and is in contact with cooling assembly/fins 620 that are, in turn, cooled by a coolant, e.g., circulating liquid nitrogen, in channels 630. This combination of coarse cooling and finer cooling/heating via the cooling transfer plate 610 can allow independent control of the temperature of each well, or with less resolution, establishment of a prescribed temperature gradient for prescribed periods of time.

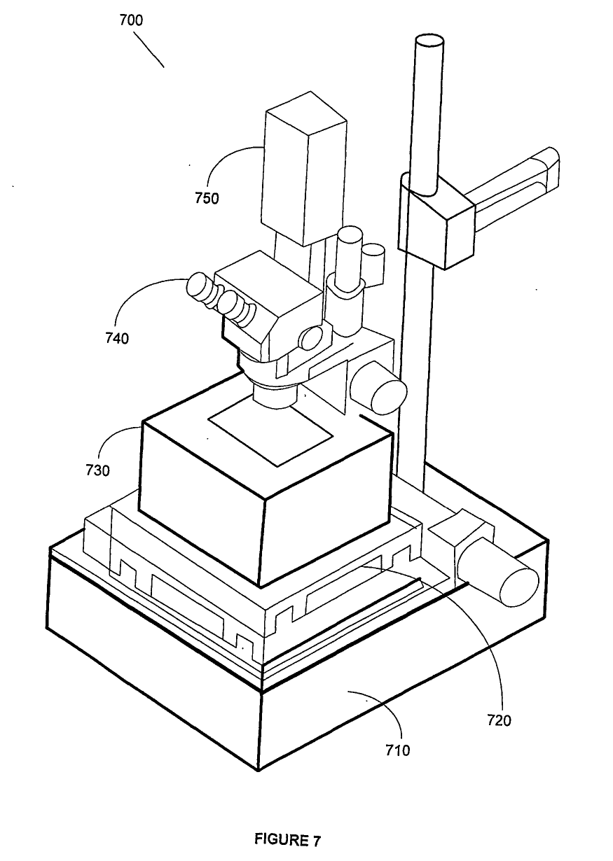

FIG. 7 illustrates the deployment of the lyophilization chamber 300 as a stage in microscope 700. Microscope base 710 supports X-Y table 720 that facilitates controlling the position of the lyophilization chamber 730. Lyophilization chamber 730 is similar to lyophilization chamber 300 shown in an exploded view in FIG. 3. One or more eyepieces 740 and camera 750 facilitate observation and recording of changes, including over time periods of interest.

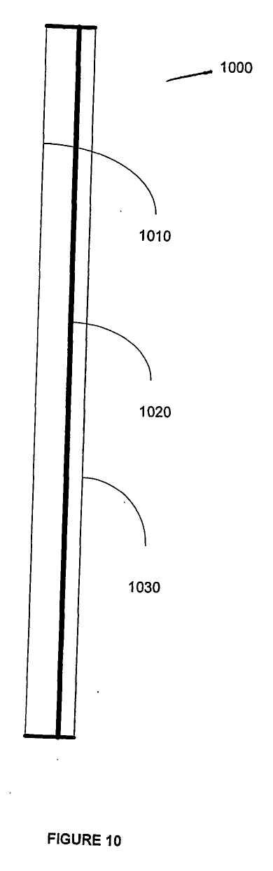

FIG. 8 shows a top view of one set of dimensions for a lyophilization plate without intending to indicate a limitation. The vapor evacuation ports 810 of the top layer are generally aligned with one end of the sample placement regions 820 of the middle layer. It should be noted that larger or smaller dimensions are possible as well. For example, the lyophilization plate length may be smaller than any single integer between 1 and 24 inches, and a width smaller than any single integer between 1 and 24 inches. A length of 5.02 inches and a width of 3.35 inches is suitable for adapting many commonly available microscopes to function with a freezing and freeze-drying stage described herein. As further shown in FIG. 9, without limitation, individual wells may have sample placement region 910 greater than 1 cm by 1 cm or as small as 1 mm by 1 mm. Included in the invention, but not limiting, are sample well placement regions with a length of any sample integer between 1 and 10 mm and a width of any single integer between 1 and 10 mm. As further shown in FIG. 9, without limitation, individual wells may have vapor evacuation port 920 greater than 1 cm by 1 cm or as small as 1 mm by 1 mm. Included in the invention, but not limiting, are sample well placement regions with a length of any sample integer between 1 and 10 mm and a width of any single integer between 1 and 10 mm. FIG. 10 further shows a side view of lyophilization plate with lyophilization plate bottom layer 1010, lyophilization plate middle layer 1020, and lyophilization plate top layer 1030. Alternative embodiments with greater height than width aspect ratios (e.g., capillary tubes) are also intended to be included within the intended scope of the invention.

The disclosure herein enables decisions to be made between excipients that otherwise are similar with a view to prepare a better freeze-dried product with a long shelf life, easy reconstitution, and low preparation cost by ensuring a rapid rate for primary and secondary drying. It further allows discovery of conditions for freeze-drying a variety of biological materials such as strains of bacteria. Moreover, the well-controlled stage also facilitates determining conditions for freezing biological materials while preserving viability. Naturally, the invention also has broad applicability in discovering methods, conditions, and components to be added for effectively and efficiently freezing and freeze-drying foodstuffs to preserve characteristics such as texture by minimizing structural damage.

In particular, the disclosed apparatus and system enables screening of samples for evaluating suitability for, or improving/optimizing, freeze-drying. In an example embodiment an array, or sub-array, of samples comprising a lyophilizable solvent is frozen, preferably by supercooling, although directional freezing may be used as well. Optionally, the samples undergo one or more freeze-thaw cycles. Sublimation is carried out by subjecting the plurality of samples to a pressure in the range defined by at least 50 micrometers of Hg to no more than 760 millimeters of Hg followed by or concurrent with examination to determine if the temperature of one or more samples has exceeded its glass transition temperature.

The frozen samples may be annealed by warming to about or below the melting point of the lyophilizable solvent and incubating for a duration of time that is preferably less than 15 hours, 10 hours, 5 hours, or 1 hour. The temperature is preferably no more than five degrees below the melting point of the lyophilizable solvent, or no more than two degrees below the melting point of the lyophilizable solvent, or no more than one degree below the melting point of the lyophilizable solvent.

Advantageously, the screening process may be used to determine a desired temperature from a range of temperatures below the melting point of the lyophilizable solvent by determining a corresponding range of primary drying times followed by selecting a temperature corresponding to a desirable primary drying time. Similarly, the secondary drying time or any combination of the primary and secondary drying times may be carried out to determine a suitable temperature for annealing.

Although the present invention has been described in considerable detail with reference to certain preferred embodiments, other embodiments are possible. Therefore, the spirit and scope of the appended claims should not be limited to the description of the preferred embodiments contained herein. Modifications and variations of the invention described herein will be obvious to those skilled in the art from the foregoing detailed description and such modifications and variations are intended to come within the scope of the appended claims.

A number of references have been cited, the entire disclosures of which are incorporated herein by reference.

1. A freeze-drying microscope stage for screening an array of samples to identify processing parameters for freeze-drying, wherein the array comprises at least 24 samples, the freeze-drying microscope stage comprises:
(a) at least one lyophilization plate comprising a plurality of stacked optically clear-layers;
(b) a plurality of chambers in said at least one lyophilization plate;
(c) at least one pressure and temperature controlled chamber having optically clear windows; and
(d) heating, cooling, and pressure controls connected to the freeze-drying microscope stage.

2. The freeze-drying microscope stage of claim 1, wherein said pressure controls enable providing a first pressure to a first sample in the array of samples and a second pressure to a second sample in the array of samples.

3. The freeze-drying microscope stage of claim 2, wherein said first pressure is not equal to said second pressure.

4. The freeze-drying microscope stage of claim 1, wherein said heating and cooling controls provide a first temperature to a first sample in the array of samples and a second temperature to a second sample in the array of samples.

5. The freeze-drying microscope stage of claim 4, wherein said first temperature is not equal to said second temperature.

6. The freeze-drying microscope stage of claim 5, wherein said heating and cooling controls enable controlling a temperature of one or more samples of the array.

7. The freeze-drying microscope stage of claim 1, wherein said heating controls enable providing heat to a surface of one or more selected samples in the array of samples.

8. The freeze-drying microscope stage of claim 1, wherein said heating controls provide volumetric heating to one or more selected samples in the array of samples.

9. The freeze-drying microscope stage of claim 6, wherein a plurality of samples in the array of samples, each sample comprising a freeze-dried fraction of a common initial formulation, correspond respectively to a plurality of temperatures which enable a determination of a glass transition temperature of the freeze-dried fraction by observing flow of the freeze-dried fraction.

10. The freeze-drying microscope stage of claim 6, wherein a plurality of samples in the array of samples are respectively maintained at a plurality of pressures whereby enabling identification of a first pressure from the plurality of pressures corresponding to a sample in the array of samples exhibiting a desired rate of freeze-drying.

11. The freeze-drying microscope stage of claim 9, wherein a structure of each of the plurality of samples in the array of samples is examined before a freeze-drying cycle.

12. The freeze-drying microscope stage of claim 9, wherein a structure of each of the plurality of samples in the array of samples is examined during a freeze-drying cycle.

13. The freeze-drying microscope stage of claim 9, wherein a structure of each of the plurality of samples in the array of samples is examined after a freeze-drying cycle.

14. The freeze-drying microscope stage of claim 6, wherein a plurality of samples in the array of samples comprise a formulation maintained at varying temperatures by the temperature control to determine a glass transition temperature of the formulation.

15. The freeze-drying microscope stage of claim 6, wherein a plurality of samples in the array of samples comprise a formulation maintained at varying temperatures by the temperature control to determine a sublimation rate of the formulation.

16. The freeze-drying microscope stage of claim 6, wherein a plurality of samples in the array of samples comprise a formulation are monitored to determine a moisture content of at least one sample in the plurality of samples.

17-40. (canceled)

41. A method of screening an array of samples for evaluating suitability for freeze-drying, comprising:
(a) preparing at least 24 samples to form the array of samples, wherein at least two samples comprise a lyophilizable solvent;
(b) freezing a plurality of samples in the array of samples;
(c) subjecting the plurality of samples to a freeze-thaw cycle by thawing and refreezing;
(d) subjecting the plurality of samples to a pressure in the range defined by at least 50 micrometers of Hg to no more than 760 millimeters of Hg; and
(e) examining, visually, at least one sample in the plurality of samples to determine if the temperature has exceeded the glass transition temperature for the sample.

42. The method of claim 41, further comprising the step of freezing a sample by supercooling.

43. The method of claim 42, further comprising the step of annealing the frozen sample by warming to about or below the melting point of the lyophilizable solvent for a duration of time.

44. The method of claim 43, wherein the step of annealing includes warming to no more than five degrees C. below the melting point of the lyophilizable solvent for a duration of time.

45. The method of claim 43, wherein the step of annealing includes warming to no more than two degrees C. below the melting point of the lyophilizable solvent for a duration of time.

46-59. (canceled)
